(19) United States
(12) Patent Application Publication
(10) Pub. No.: US 2023/0070155 A1
(43) Pub. Date: Mar. 9, 2023

(54) Design, Fabrication, and Characterization of Nanoplastics and Microplastics

(71) Applicant: Research Triangle Institute, Research Triangle Park, NC (US)

(72) Inventors: Ninell Mortensen, Holly Springs, NC (US); Leah Johnson, Durham, NC (US); Jeffrey Brent Mecham, Chapel Hill, NC (US)

(21) Appl. No.: 17/798,188

(22) PCT Filed: Feb. 19, 2021
(86) PCT No.: PCT/US2021/018695

§ 371 Date: Aug. 8, 2022

Related U.S. Application Data
(60) Provisional application No. 62/978,499, filed on Feb. 19, 2020.
(60) Provisional application No. 63/089,210, filed on Oct. 8, 2020.

(51) Int. Cl. C09K 11/02; G01N 21/64; C09K 11/06; G01N 33/58; G01N 33/543; B29B 9/12

(52) U.S. Cl. CPC C09K 11/02; G01N 21/6428; C09K 11/06; G01N 33/582; G01N 33/54346; B29B 9/12; G01N 2021/6439; B29K 2067/003

(57) ABSTRACT

Provided by the inventive concept or nanoplastic or microplastic particles, reference standard materials including nanoplastic or microplastic particles, methods of using, and methods of preparing the same. Uses of the nanoplastic and/or microplastic particles of the inventive concept include tracking of nanoplastic and/or microplastic particle dispersion/distribution in environmental and/or biological systems, as well as in organisms that are within the environment.

### CROSS-REFERENCE TO RELATED APPLICATIONS

This application claims the benefit of U.S. Provisional Application Ser. No. 62/978,499, filed Feb. 19, 2020, and U.S. Provisional Application Ser. No. 63/089,210, Oct. 8, 2020, the entireties of each of which is incorporated herein by reference.

### BACKGROUND

A critical need exists to evaluate the presence and downstream effects of nano- and microplastics in the environment and within biological systems. Despite the escalating magnitude of this issue, commercially available and well characterized nano- and microplastics are severely limited (e.g., primarily polystyrene), which restricts crucial advancements in understanding human health and environmental effects. For example, the importance of well-characterized standards in nanotechnology,—medicine, and—toxicology has been emphasized in the literature for more than a decade¹⁻⁵.

The reliance of society on plastics is evident from global production, which reached over 330 million tons in 2016. Although plastics are undeniably beneficial, widespread utilization has resulted in an unforeseen issue: an abundance of unintentional plastic debris, including nanoplastics and microplastics, in the environment. An estimated 4.8 to 12.7 million metric tons of plastic debris entered the world's oceans during 2010. In September 2017, microplastic were reported in 94% of tested tap water samples in the United States and were found in 93% of tested bottled water samples in March 2018.

Nanoplastics and microplastics can infiltrate, often undetected, through the environment and into biological systems and products. Microplastics have been found in shellfish, mussels, fish, and products including honey, sea salt, as well as drinking water and beverages. These nanoplastics and microplastics also can leach exogenous chemicals, such as formulation additives or unreacted monomers. Many plastic related chemicals, found in drinking water and food products, are known toxicants in human health and the human health risk of unintentional exposure to nanoplastics and microplastics and associated chemicals is unknown.

Accordingly, there is a need for development of compositions/materials, and methods of using such compositions/materials, for the tracking of nanoplastics and microplastics in organisms and in the environment.

### SUMMARY

According to an aspect of the inventive concept, provided is a nanoplastic or microplastic particle including: a nanoplastic or microplastic polymer, polymer composite, or polymer matrix; and a fluorescent tag or a radioactive tag.

According to another aspect of the inventive concept, provided is a reference standard material including nanoplastic or microplastic particles, the nanoplastic or microplastic particles including: a nanoplastic or microplastic polymer, polymer composite, or polymer matrix; and a fluorescent tag or a radioactive tag.

According to still another aspect of the inventive concept, provided is a method of monitoring environmental dispersion of nanoplastic or microplastic particles including: providing the reference standard material of the present inventive concept to an environment; and monitoring dispersion of the reference material in the environment, wherein monitoring dispersion of the reference material includes detecting presence of the reference material in at least one sample from the environment.

According to yet another aspect of the inventive concept, provided is a method of monitoring dispersion of nanoplastic or microplastic particles in a subject including: exposing the subject to the reference standard material of the inventive concept; and monitoring dispersion of the reference material in the subject, wherein monitoring dispersion of the reference material includes detecting presence of the reference material in at least one sample from the subject.

According to yet another aspect of the inventive concept, provided is a method of monitoring the presence of nanoplastic or microplastic particles in a sample including: providing a reference material including a nanoplastic or microplastic particles, the nanoplastic or microplastic particles including a polymer, polymer composite or polymer matrix, and a fluorescent tag or a radioactive tag to an environment; and determining whether the reference material is present in a sample obtained from the environment.

According to yet another aspect of the inventive concept, provided is a method of preparing nanoplastic or microplastic particles including: dissolving a plastic in a first solvent to provide a plastic solution; precipitating the plastic solution in a second solvent; and evaporating the first solvent to provide a dispersion of the nanoplastic or microplastic particles in the second solvent.

### BRIEF DESCRIPTION OF THE DRAWINGS

FIG. 1: Architectures of nanoplastic or microplastic particles—(A) Solid, (B) Matrix, (C-D) Core Shell Functionalized with Tracer (C) or Chemical Groups (D).

FIG. 2: SEM of polyethylene terephathalate (PET) nanoplastic particles (148 nm) according to embodiments of the present inventive concept.

FIG. 3: Fluorescence images of (panel A) PET nanoplastics containing rhodamine-B (RB) and (panel B) PET nanoplastics containing fluorescein visualized on BeWo trophoblast b30 cells (nuclei are stained blue).

FIG. 4: Fluorescence images of PET-RB and polystyrene (PS) Alexa Fluor (AF)488 nanoparticles visualized on BeWo trophoblast b30 cells (nuclei are stained blue).

FIG. 5: MTS assays examining the cytotoxicity of PET and PS nanoplastic particles. PET nanoplastics exhibited a cytotoxicity, as determined by MTS assays measuring metabolic activity.

FIG. 6: Exemplary PET nanoplastic particles prepared as described in EXAMPLE 3.

FIG. 7: (panel A) an SEM image, (panel B) a TEM image, and (panel C) a DLS curve for PET-RB NPs.

FIG. 8: FT-IR spectra for (top) PET NPs and (bottom) PET-RB NPs.

FIG. 9: Cytotoxicity of PET-NP (black) and PET-RB NPs (gray) tested by (panel A) membrane integrity (LHD release) and (panel B) metabolic activity (MTS assay). The graphs show mean±standard deviation. One asterisk shows P-values <0.05 and two asterisks show P-values <0.001.

FIG. 10: (panels A−D) bright field, and (panels E−H) fluorescence microscopy of RAW 264.7 cells exposed to control (panels A+E), 0.005 mg/mL (panels B+F), 0.05 mg/mL (panels C+G), and 0.5 mg/mL PET-RB NPs (panels D+H). The images from individual fluorescence channels are shown in FIG. 14. Cell nuclei appear on the blue channel, cell cytoplasm on the green channel, and PET-RB NP on the red channel.

FIG. 11: FT-IR spectra of the PET starting material.

FIG. 12: Raman spectra of PET NPs and PET-RB NPs in 0.5 mg/mL of BSA.

FIG. 13: Pyrolysis-GC/MS chromatograms of (top) the PET fiber used for fabrication of (middle) PET-NP and (bottom) PET-RB NP. Four of the characteristic peaks were identified as (1) vinyl benzoate, (2) benzoic acid, (3) divinyl terephthalate, and (4) 4-(vinyloxycarbonyl benzoic acid).

FIG. 14: Fluorescence microscopy of RAW 264.7 cells exposed to PET-RB NPs, showing the overlay images of the three fluorescence channels (panels A−D), PET-RB NPs (panels E−H), cell cytoplasm (panels I−L), and nuclei (panels M−P) for control (panels A+E+I+M), 0.005 mg/mL PET-RB NPs (panels B+F+J+N), 0.05 mg/mL (panels C+G+K−O), and 0.5 mg/mL PET-RB NPs (panels D+H+L+P).

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

The term “comprise,” as used herein, in addition to its regular meaning, may also include, and, in some embodiments, may specifically refer to the expressions “consist essentially of” and/or “consist of.” Thus, the expression “comprise” can also refer to, in some embodiments, the specifically listed elements of that which is claimed and does not include further elements, as well as embodiments in which the specifically listed elements of that which is claimed may and/or does encompass further elements, or embodiments in which the specifically listed elements of that which is claimed may encompass further elements that do not materially affect the basic and novel characteristic(s) of that which is claimed. For example, that which is claimed, such as a composition, formulation, method, system, etc. “comprising” listed elements also encompasses, for example, a composition, formulation, method, kit, etc. “consisting of,” i.e., wherein that which is claimed does not include further elements, and a composition, formulation, method, kit, etc. “consisting essentially of,” i.e., wherein that which is claimed may include further elements that do not materially affect the basic and novel characteristic(s) of that which is claimed.

The term “about” generally refers to a range of numeric values that one of skill in the art would consider equivalent to the recited numeric value or having the same function or result. For example, “about” may refer to a range that is within ±1%, ±2%, ±5%, ±10%, ±15%, or even ±20% of the indicated value, depending upon the numeric values that one of skill in the art would consider equivalent to the recited numeric value or having the same function or result. Furthermore, in some embodiments, a numeric value modified by the term “about” may also include a numeric value that is “exactly” the recited numeric value. In addition, any numeric value presented without modification will be appreciated to include numeric values “about” the recited numeric value, as well as include “exactly” the recited numeric value. Similarly, the term “substantially” means largely, but not wholly, the same form, manner or degree and the particular element will have a range of configurations as a person of ordinary skill in the art would consider as having the same function or result. When a particular element is expressed as an approximation by use of the term “substantially,” it will be understood that the particular element forms another embodiment.

Unless otherwise defined, all technical and scientific terms used herein have the same meaning as commonly understood by one of ordinary skill in the art to which this invention belongs.

### Compositions

Embodiments of the inventive concept include engineered nanoplastic and/or microplastic particles that have been chemically designed and processed into forms that are capable of use as reference standard materials; we have demonstrated the ability to use these materials in biological systems.

The material of the nanoplastic and microplastic particle can be a polymer, a polymer composite or a polymer matrix. In some embodiments, the nanoplastic and/or microplastic particle includes polyethylene terephathalate (PET), polyethylene (PE), high density PE (HDPE), low density PE (LDPE), linear-low-density polyethylene (LLDPE), polyvinyl chloride (PVC), polypropylene (PP), polystyrene (PS), polylactic acid (PLA), polycarbonate (PC) polymethyl methacrylate (PMMA), Polyamide (PA), polyacrylic acid (PAA), polyacrylonitrile (PAN), polyoxymethylene (POM), polyurethane (PUR), silicone, nylon, or acrylonitrile butadiene styrene (ABS). In some embodiments, the polymer, a polymer composite or a polymer matrix includes PET. In some embodiments, the polymer, a polymer composite or a polymer matrix includes PS.

In some embodiments, the nanoplastic and microplastic particles are prepared by bottom-up approaches. In some embodiments, the nanoplastic and microplastic particles are prepared by top-down approaches. Methods for preparing nanoplastic and microplastic particles include, but are not limited to, self-assembly, condensation, nucleation, colloidal methods, sol-gel processing, micromulsion of oil-water, hydrothermal synthesis, polyol method, sonochemical approaches, emulsion polymerization, dispersion polymerization, and microemulsion polymers. In certain embodiment the particle is prepared by chain growth polymerization. Nonlimiting examples of chain growth polymerization for preparing particles include radical chain polymerization, anionic chain polymerization, and cationic chain polymerization. In one nonlimiting example, the material of the particle is prepared using radical chain polymerization of monomers containing one or more acrylate or vinyl functionalities.

The particles can be prepared using chemical processes, physico-chemical processes, physico-mechanical processes, or combinations thereof. Nonlimiting examples of chemical processes for preparing particles includes suspension polymerization, emulsion polymerization, dispersion polymerization, polycondensations polymerization and combinations thereof.

Nonlimiting examples of physico-chemical processes for preparing particles includes coacervation, layer-by-layer assembly, sol-gel encapsulation, supercritical CO₂ encapsulation, and combinations thereof. Nonlimiting examples of physico-mechanical processes for preparing particles includes spray drying, multiple nozzle drying, fluid bed coating, centrifugal techniques, vacuum encapsulation, electrostatic encapsulation, and combinations thereof. In some embodiments, a core-shell particle is formed by an interfacial reaction between two immiscible monomers at the interface between the core and surrounding solution.

Methods of preparing nanoplastic and/or microplastic particles of the inventive concept may include dissolving a plastic in a first solvent to provide a plastic solution; precipitating the plastic solution in a second solvent; and evaporating the first solvent to provide a dispersion of nanoplastic or microplastic particles in the second solvent. Methods/techniques of dissolving, precipitating, and/or evaporating are not particularly limited, and may be performed using any method/technique that may be appreciated by one of skill in the art.

In some embodiments, the plastic may be any one of, but not limited to, polyethylene terephthalate (PET), polyethylene (PE), high density PE (HDPE), low density PE (LDPE), linear-low-density polyethylene (LLDPE), polyvinyl chloride (PVC), polypropylene (PP), polystyrene (PS), polylactic acid (PLA), polycarbonate (PC) polymethyl methacrylate (PMMA), Polyamide (PA), polyacrylic acid (PAA), polyacrylonitrile (PAN), polyoxymethylene (POM), polyurethane (PUR), silicone, nylon, or acrylonitrile butadiene styrene (ABS), or any combination thereof In some embodiments, the plastic is PET. In some embodiments, the first solvent may be any one of, but not limited to, phenol, DMSO, nitrobenzene, o-chlorophenol, o-cresol, diphenylamine, dichloromethane, or HFIP, or any combination thereof In some embodiments, the solvent is HFIP. In some embodiments, the plastic solution may include the plastic at a concentration/in an amount between about 0.1 weight % and about 0.5 weight %, but is not limited thereto. In some embodiments, the second solvent may be, but is not limited to, water.

Precipitating of the plastic solution may be performed, for example, precipitating the plastic solution in the second solvent by adding the plastic solution to the second solvent at a rate, for example, but not limited to, about 0.1 mL/min and about 5 mL/min. In some embodiments, the plastic solution is added to the second solvent at a rate of about 1 mL/min.

Volumes and temperatures of the solutions/solvents used in dissolving and/or precipitating according to methods of preparing nanoplastic and/or microplastic particles of the inventive concept may be any volume and/or temperature envisioned by one of skill in the art to perform the methods of the inventive concept. For example, the plastic solution may have a volume of about 10 mL, and the second solvent may have a volume of between about 50 mL and about 5000 mL, and the second solvent may have a temperature of about 0° C. and about 20 ° C.

The particle of the inventive concept can be modified to enable monitoring of the particle through biological material. In some embodiments, the plastic particle contains fluorescent tag distributed through the polymer matrix. Nonlimiting example of fluorescent tags include rhodamine, such as rhodamine-B (RB), fluorescein, Alexa-Fluor compounds, Nile Red, R-Phycoerthyrin, Pacific Blue, Cascade Blue, Texas Red, Cy5, Cy3, Cy7, hydroxycoumarin, aminocourmarin, methoxycoumarin, and the like. In one nonlimiting example, the fluorescent compound is a bioconjugate. In other embodiments, the particle may have a radioactive tag or label, for example, but not limited to ¹⁴C, or ³H. Particles of the inventive concept modified as described herein may be prepared by, for example, dissolving a plastic in a first solvent with, for example, a fluorescent tag, such as described herein.

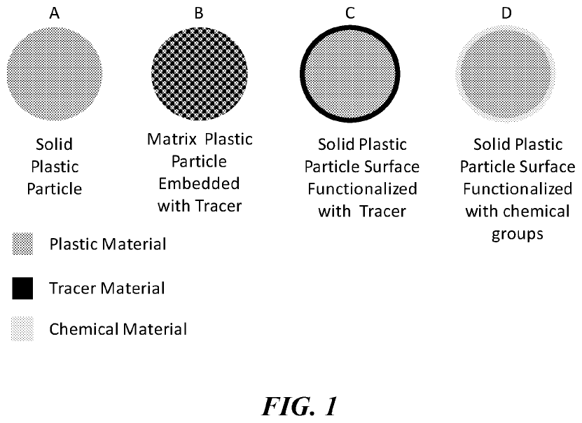

The architecture of the labeled particle system includes solid, matrix or surface-functionalized (FIG. 1). In one nonlimiting example, the nanoplastic particle is a matrix style with fluorescent tracer distributed throughout the polymer matrix. In another nonlimiting example, the nanoplastic particle is surface-functionalized, with the fluorescent tracer associated with the surface of the particle. Surface-functionalization can also include chemical groups.

Nonlimiting example of such chemical groups are —COOH, —COO⁻—NH₃⁺, —NH₂, —OH, —PEG, streptavidin, a streptavidin-biotin complex, antibodies and the like. Nonlimiting examples of nanoplastic or microplastic particle morphology according to embodiments of the present inventive concept include spheres, fibers, rods, and dendrimers.

In some embodiments, the particle size or average particle size is less than about one micron, less than about 0.9 microns, less than about 0.8 microns, less than about 0.7 microns, less than about 0.6 microns, less than about 0.5 microns, less than about 0.4 microns, less than about 0.3 microns, less than about 0.2 microns, or less than about 0.1 microns. In some embodiments, the particle size or average particle size is less than 500 nm. In some embodiments, the particle size or average particle size is less than 200 nm. In some embodiments, the particle size or average particle size is less than 150 nm. In some embodiments, the particle size or average particle size is less than 100 nm. In some embodiments, the particle of the inventive concept is sized to represent particle size distributions of nanoplastic and/or microplastic particles found in the environment.

In some embodiments, provided are fabricated nanoplastic and/or microplastic particles, for example, PET nanoplastic particles. The PET particle system of the present inventive concept can remain in aqueous suspension that permits use in biological systems.

### Methods

In other embodiments of the present inventive concept, provided are methods for monitoring presence and/or dispersion of nanoplastics or microplastics, such as nanoplastic particles or microplastic particles dispersed in, for example, an environment, a biological system, and/or a lifeform. The nature of the method is not particularly limited, and may be any method for monitoring that may be appreciated by one of skill in the art. For example, the method for monitoring nanoplastics or microplastics may be an in vitro, in situ, in vivo, or ex vivo method without departing from the spirit of the present disclosure. Monitoring for presence and/or dispersion of nanoplastics or microplastics may include providing or obtaining a sample from an environmental or biological system, and qualitatively or quantitatively determining/detecting if nanoplastics or microplastics are present in the sample.

The nature of the environmental or biological system is not particularly limited. For example, the environmental or biological system may be a marine, freshwater, or terrestrial environment, or a marine, freshwater, or terrestrial biological system. Included among the biological systems may be biological lifeforms, for example, marine, freshwater, or terrestrial lifeforms. The lifeforms may be single cell or multicellular and may be plant or animal lifeforms without departing from the scope of the present inventive concept. In some embodiments, the animal lifeform may be a mammalian lifeform, without limitation, e.g., a rodent, primate, or human lifeform. The presence and/or dispersion of nanoplastics or microplastics may be monitored by any, for example, in vitro, in situ, in vivo, or ex vivo method that would be appreciated by one of skill in the art, or any combination thereof In some embodiments, samples may be drawn from a lifeform may include, but are not limited to, fecal or waste samples, organ or tissue samples and/or placental samples, which may be analyzed for the presence of and/or dispersion of nanoplastics and/or microplastics. In some embodiments, the environmental or biological systems may include, soil, sediment, or water, from which samples may be drawn and analyzed for the presence of and/or dispersion of nanoplastics and/or microplastics. In some embodiments, samples from food products and/or consumer products may be drawn and analyzed for the presence of and/or dispersion of nanoplastics and/or microplastics.

Methods for monitoring presence and/or dispersion of nanoplastics and/or microplastics may include analytical methods, such as, for example, high-resolution pyrolysis GC-MS and the like. In some embodiments, monitoring for presence and/or dispersion of nanoplastics and/or microplastics may include tracking fluorescence emitted by fluorescently labeled nanoplastic and/or microplastic reference standard materials as described herein. In other embodiments, monitoring for presence and/or dispersion of nanoplastics and/or microplastics may include tracking radioactivity emitted by radioactively labeled nanoplastic and/or microplastic reference standard materials as described herein.

Having described various aspects of the present invention, the same will be explained in further detail in the following examples, which are included herein for illustration purposes only, and which are not intended to be limiting to the invention.

### EXAMPLE 1

### Fabrication of Pet Nanoplastic Particles

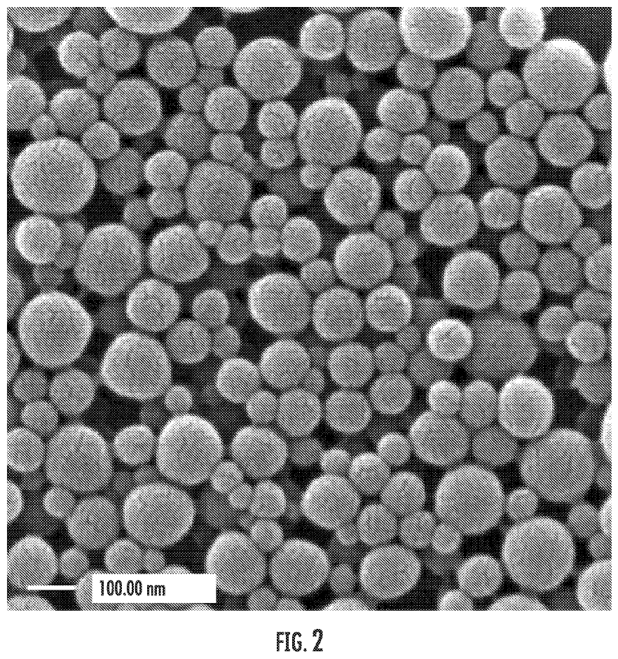

A solution of PET was made from PET fiber and hexafluoroisopropanol (HFIP). The solution was then precipitated into chilled DI water (i.e., 7:1 ratio of non-solvent to solvent) at 0° C. in a beaker. The entire contents of the precipitation vessel were then rotary evaporated under vacuum at 37° C. to distill off any remaining HFIP. The water-dispersed PET nanoplastic particles were collected by centrifugation. Nanoplastic or microplastic particles were imaged either by SEM or lightfield microscope. The hydrodynamic diameter was characterized by Dynamic Light Scattering (DLS, Malvern Zetasizer Nano-ZS, Malvern Panalytical). The diameter of microplastic particles were measured using a Mastersizer 2000 (Malvern Zetasizer Nano-ZS, Malvern Panalytical). A scanning electron micrograph (SEM) of 148 nm PET nanoplastic particles, prepared as described herein, is exemplified in FIG. 2.

### EXAMPLE 2

### Fabrication of Pet Nanoplastic Particles Encapsulated With a Flourscent Tracer

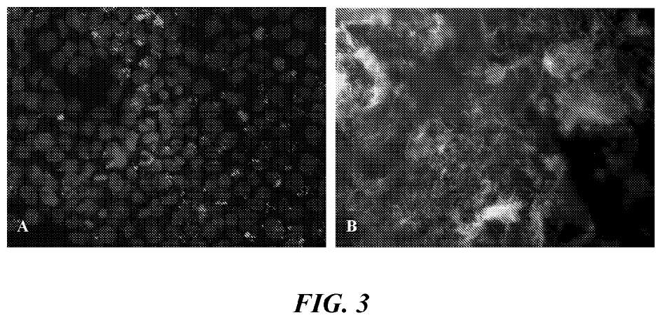

A solution of PET was made from PET fiber and hexafluoroisopropanol (HFIP). Formulations contained trace quantities of either fluorescein or rhodamine B. The solution was then precipitated into chilled DI water (i.e., 7:1 ratio of non-solvent to solvent) at 0° C. in a beaker. The entire contents of the precipitation vessel were then rotary evaporated under vacuum at 37° C. to distill off any remaining HFIP. The water-dispersed PET nanoplastic particles were collected by centrifugation. The PET nanoplastic particles are imaged by a fluorescence microscope (FIG. 3).

### EXAMPLE 3

### Biological Effects of Nano- And Microplastics Related to Human Health

Microplastics have been found in shellfish, mussels, fish, and products including honey, sea salt, as well as drinking water and beverages. The health effects of microplastics present in the environment and consumer products are unknown.

### Objectives

The objective of this project is to investigate how ingested nanoplastic and microplastic particles (NMPs) and the accompanying plastic-related exogenous chemicals released from such particles (e.g., plasticizers and contaminants) interact with biological systems in vitro and in vivo. The goal is to investigate risk on human health that is associated with exposure to these complex materials. We hypothesize that both the NMPs and the released plastic-related chemicals will impact biological systems following ingestion. Therefore, exposure studies of NMPs differ from exposure studies of other nano- and micromaterials because equal attention to the fate of the particle and the fate of related chemicals is required.

### Methods

### Fabrication of PET Nanoplastic Particles

A 1.67% (v:v) solution of PET was prepared by mixing 0.25 g PET fiber and 15 mL hexafluoroisopropanol (HFIP, CAS#920-66-1) in a scintillation vial with an 0.5″ stir bar.

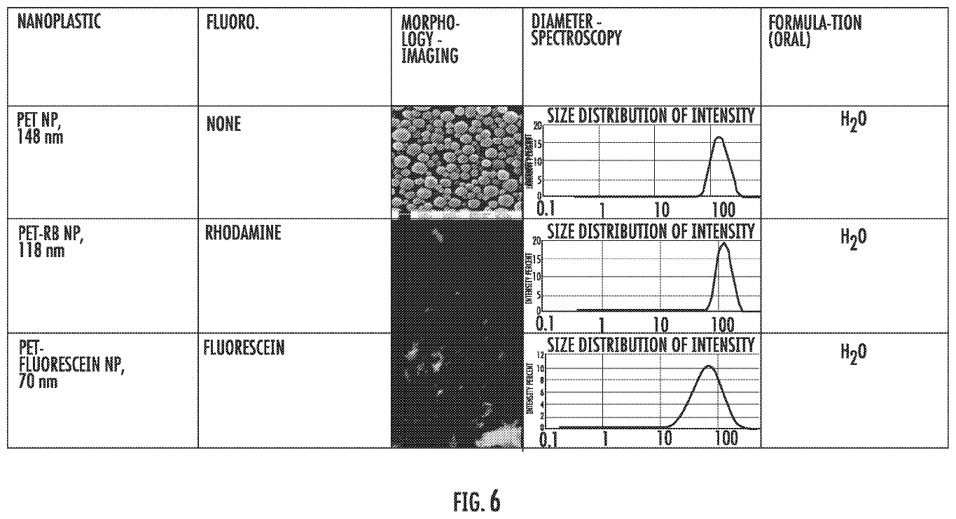

Formulations containing either fluorescein or rhodamine B were prepared using the same approach, plus the addition of the dye at a concentration of 0.0001 weight percent. The formulations were then stirred at 600 rpm for 10 minutes to afford a clear solution, or colored solutions when dyes were included. Each solution was then precipitated into 105 ml chilled DI water (i.e., 7:1 ratio of non-solvent to solvent) at 0° C. in a 500 mL beaker. The chilled DI water was stirred rapidly with a 2″ magnetic stir bar and the HFIP solution was added dropwise to produce a cloudy dispersion of particles. The entire contents of the precipitation vessel were then rotary evaporated under vacuum at 37° C. to distill off any remaining HFIP. The water-dispersed PET nanoparticles collected by centrifugation at 4000 g for 10 minutes resulting in a pellet of dense particles at the bottom of 50 ml centrifuge tube. Most of the water was then decanted off and the slurry was analyzed using scanning electron microscopy and DLS analysis to determine particle size and polydispersity. PET nanoplastic particles prepared as described above are shown in FIG. 6.

### Nanoplastic and Microplastic Particle Characterization

Nanoplastic and microplastic particles were imaged either by SEM or fluorescence microscopy. The hydrodynamic diameter was characterized by Dynamic Light Scattering (DLS, Malvern Zetasizer Nano-ZS, Malvern Panalytical). The diameter of microplastic particles was measured using a Mastersizer 2000 (Malvern Zetasizer Nano-ZS, Malvern Panalytical).

### Results

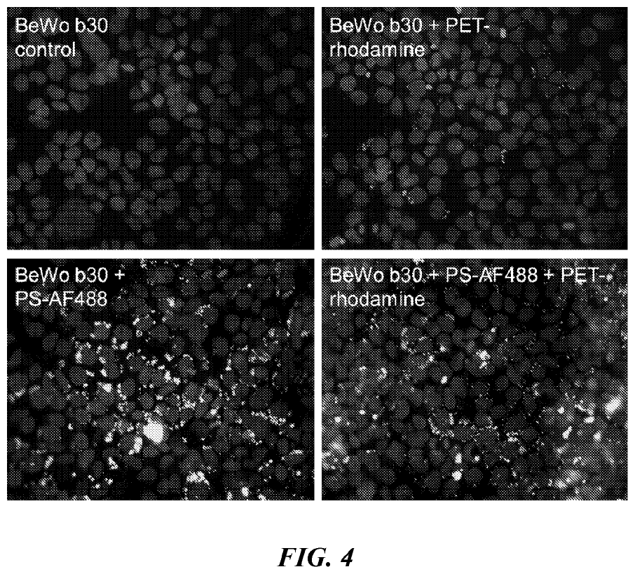

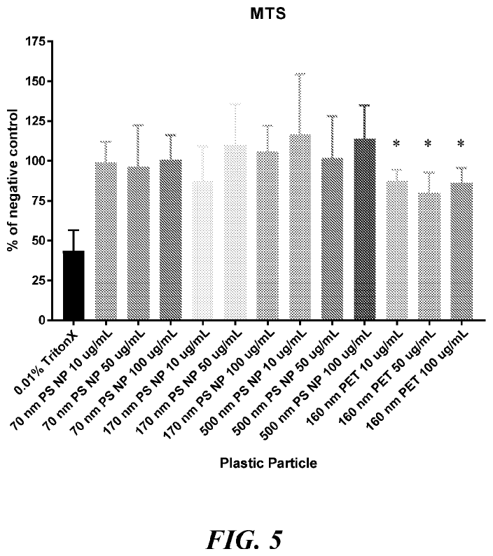

Creating a library of nano- and microplastic particles was initiated by fabricating and procuring materials. Each material was characterized and formulated in a vehicle suitable for oral administration to laboratory animals. FIG. 4 shows imaging of PET-RB NP on BeWo b30 cells. FIG. 5 depicts MTS assays measuring metabolic activity in trophoblast cells exposed to PET nanoplastic particles and PS nanoplastic particles. PET nanoplastic particles were observed to induce a cytotoxic response, whereas PS nanoplastic particles did not.

### Conclusions

### PET Fabrication and Plastic Particle Library

PET nanoplastic particles and nanoplastic fibrils with and without contrast agents have been successfully fabricated.
A plastic particle library to capture the breath of benchmark reference materials has been initiated.

### Biological Impact of Nanoplastic Particles and Associated Chemicals

Fluorescence microscopy images indicate that PET and PS nanoplastic particles are taken up by the trophoblast cells (FIG. 4).
PET nanoplastic particles induced a cytotoxic response in trophoblast cells, while PS nanoplastic particles did not (FIG. 5).

### Significance

The federal and public interest in nanoplastics and microplastics and their potential health impact is rapidly increasing. The federal agencies are emphasizing the need for validated nanoplastics and microplastics detection and characterization methods and standards:
The Joint Group of Experts on the Scientific Aspects of Marine Environmental Protection (GESAMP), 2010: Knowledge of the distribution and fate of microplastics is only beginning to emerge.¹
European Food Safety Authority (EFSA), 2016: Published report: “Presence of microplastics and nanoplastics in food, with particular focus on seafood”, and concluding that: Research on the toxicokinetics and toxicity, including studies on local effects in the gastrointestinal (GI) tract, are needed as is research on the degradation of microplastics and potential formation of nanoplastics in the human GI tract.²
United States Environmental Protection Agency (EPA), 2017: Held Microplastic Expert Workshop in June 2017, focusing on four areas 1) Method needs, 2) Microplastics sources, transport and fate needs, 3) Ecological assessment needs, and 4) Human health assessment needs.³
World Health Organization (WHO), 2019: “The World Health Organization (WHO) today calls for a further assessment of microplastics in the environment and their potential impacts on human health, following the release of an analysis of current research related to microplastics in drinking-water”.⁴
The National Science Foundation (NSF)—Topics for FY 2020 Emerging Frontiers in Research and Innovation (NSF 19-599), 2019: “Engineering the Elimination of End-of-Life Plastics (E3P): . . . Their inherent durability leads to ever-increasing accumulation in landfills and the environment, where they eventually fragment into microplastics that contaminate waterways, wildlife, and human bodies.”
National Toxicology Program (NTP, presentation at workshop) October 2019: What nanoplastics are present in the environment and microplastics.
Food and Drug Administration (FDA what are we exposed to? If we can detect and analyze nanoplastics we can detect and analyze, presentation at NSF workshop) Dececember 2019: There is a need for validated methods and standards in detection and characterization of microplastics.

Currently there is a lack of benchmark nanoplastics and microplastics which propose a challenge for developing validated detection and characterization methods. A limitation that is being addressed by fabrication of nanoplastic and microplastic particles as described herein.

### References

1. GESAMP, Proceedings of the GESAMP International Workshop on Microplastic particles as a vector in transporting persistent, bioaccumulating and toxic substances in the ocean. 2010, The Joint Group of Experts on the Scientific Aspects of Marine Environmental Protection
2. EFSA, Presence of microplastics and nanoplastics in food, with particular focus on seafood. EFSA Journal 2016. 14(6): p. 4501.
3. EPA, Microplastics Expert Workshop Report—Trash Free Waters Dialogue Meeting. 2018.
4. WHO, Microplastics in drinking-water. 2019.

### EXAMPLE 4

### Fabrication of Polyethylene Terephthalate (Pet) Nanoparticles With Fluorescent Tracers for Studies in Mammalian Cells

Herein, the synthesis of PET NPs with a tight size distribution using a facile, bottom-up fabrication approach is reported. It is further shown that incorporation of fluorescent tracers into the NPs enables visualization and characterization of these PET NPs within mammalian cells.

### Materials and Methods

### Fabrication of PET NPs.

A solution of PET was prepared by mixing 0.58 g PET fiber (IZO Home Goods) with 35 mL hexafluoroisopropanol (HFIP) (Sigma-Aldrich, St. Louis, Mo., USA) in a 40-mL scintillation vial equipped with a magnetic stir bar. PET solution (10 mL) was added dropwise at 1 mL/min using a syringe pump (Model # NE-300, New Era Pump Systems, Inc., Farmingdale, N.Y., USA) with a Poulten & Graf GmbH Fortuna® Optima® 10-mL glass syringe into ultrapure deionized water (75 mL, 18.2 MΩ·cm resistivity) at room temperature, resulting in precipitation of PET NPs. The entire contents of the precipitation vessel were transferred to a 250-mL round-bottomed flask and rotary evaporated under vacuum at 55° C. to remove residual HFIP. Upon reduction of the volume in the round-bottomed flask (˜30 mL), ultrapure deionized water (˜75 mL) was added and the flask was subjected to rotary evaporation for a second time. The concentrated suspension of particles was pipetted into a 20-mL scintillation vial. Particles containing Rhodamine B (Sigma-Aldrich, St. Louis, Mo., USA) were formulated using a similar approach as specified above. The tracer solution in HFIP (0.05 mg/mL) was prepared from a stock solution of 1 mg/mL. An aliquot of the 0.05 mg/mL tracer solution (1 mL) was then added to the PET solution prior to precipitation into ultrapure deionized water.

To remove residual HFIP, the suspension of particles was centrifuged and resuspended.

Each wash step consisted of centrifuging the suspension at 13.1 rpm for 5 minutes at room temperature, removing the supernatant, and resuspending in an equal volume of 0.5 mg/mL Bovine Serum Albumin (BSA) to maintain the concentration of the particles in suspension. The particles were resuspended by a 30 second vortex step followed by discrete sonication in a cup horn sonicator (Ultrasonic Liquid Processor S-400, Misonic Inc., Farmingdale, N.Y.) delivering a total of 840 J/mL. For the first wash step, the initial particle suspension was spiked with BSA to a final concentration of 0.5 mg/mL before the first centrifuge step. The particles were washed three times. After the last resuspension, the hydrodynamic diameter of the particles was measured by Dynamic Light Scattering (DLS) (Malvern Zetasizer Nano-ZS, Malvern Panalytical, Westborough, Mass.). The Zeta potential (Malvern Zetasizer Nano-ZS, Malvern Panalytical, Westborough, Mass.) was measured using disposable Folded Capillary Zeta Cells

(Malvern Panalytical, Westborough, Mass.). The suspension of particles that were used for FT-IR and Pyrolysis Gas Chromatography/Mass Spectrometry (Pyro-GC/MS) were washed using water instead of 0.5 mg/mL BSA. To determine the concentration of particles, an aliquot (1 mL) of PET particles was transferred to a tared 2-mL Eppendorf tube and placed in a vacuum oven under ambient conditions overnight. The tube was weighed the next day to determine the dry particle weight. To determine the concentration of rhodamine-B within the particles, dried particles were subsequently dissolved in HFIP (1 mL) and their fluorescence was determined using Synergy MX multi-mode plate reader (BioTek Instruments, Inc, Winooski, Vt., USA). A calibration curve of Rhodamine B in HFIP, obtained via serial dilutions of the fluorophore (1.25 μg/mL stock solution, λₑₓ=550 nm, λₑₘ=580 nm).

### Characterization of PET NPs.

Fourier-transform infrared spectroscopy (FT-IR): The dried samples were analyzed with a Nicolet 6700 FTIR with a Smart Orbit™ single bounce diamond crystal ATR accessory. The instrument has a DTGS detector and a KBr beam splitter. Method parameters were set at resolution of 4 and 32 scans, scanning the region 4000-400 cm⁻¹. A background was run on the cleaned crystal before each sample. After the background acquisition was complete, a small amount of sample was added to the diamond crystal, pressure was applied, then data was acquired.

¹⁹F nuclear magnetic resonance spectroscopy (₁₉F NMR): The presence of residual hexafluoro-2-propanol within the PET NPs was determined by ¹⁹F-NMR. The fluorine NMR experiments were performed on a Varian Unity Inova 500 mHz NMR (Palo Alto, Calif.) with a Nalorac Cryogenics Corporation dedicated H-F observed probe (Martinez, Calif.). ¹⁹F-NMR samples were mixed with D20 at 10 percent. Total recycling time was 8 seconds. An external reference standard was used to calibrate and quantitate the remaining fluorine using Agilent VnmrJ ver. 4.2 software (Santa Clara, Calif.) with a limit of detection of 0.02 mM.

Transmission Electron Microscopy (TEM): PET NPs were prepared using the drop mount method for liquid deposition. PET NPs were pipetted onto 200 mesh carbon coated copper transmission electron microscopy (TEM) grids. The liquid suspension was dried in air on the copper grids inside a HEPA filtered fume hood. Two TEM grids were prepared per sample. The grids were analyzed using a Hitachi H-7000 transmission electron microscope. Multiple images were taken of each sample using an AMT digital camera. Analytical magnifications ranged between 40,000× to 300,000×.

Scanning Electron Microscopy (SEM): SEM was performed using a Zeiss Auriga field emission scanning electron microscope (FESEM) (Carl Zeiss Microscopy, White Plains, N.Y.) at 5 kV accelerating voltage and a beam current of 10 μA. Prior to SEM analysis, all samples were sputter coated with Au/Pd. The particle diameter was measured using ImageJ (NIH).

X-ray photoelectron spectroscopy (XPS): Measurements were carried out on an Escalab Xi+XPS (Thermo Fisher Scientific, Waltham, Mass.). All scans were charge compensated. Survey scans were run at 200 eV pass energy with 1.0 eV step size and 10 ms dwell time. While single element scans were done at 50 eV pass energy with 0.1 eV step size and 50 ms dwell time.

Raman Spectroscopy: The spectra of all samples were measured at room temperature using a Horiba XploRA Raman Confocal Microscope (Horiba Scientific, Piscataway, NJ) at wavelength excitation of 532 nm with 1200 L mm-1 grating.

Ultraviolet-Visible Spectroscopy (UV-VIS): Samples were analyzed using a Shimadzu UV-2600 UV-Visible Spectrophotometer (Columbia, Md.) with LabSolutions software, version 1.03 (Atlanta, Ga.) at a wavelength range of 200 to 800 nm. Samples were diluted 1:10 and 1:100 in BSA, and BSA was used as the blank. A slit width of 2 nm was used with a data interval of 0.5 nm.

Pyrolysis Gas Chromatography/Mass Spectrometry (Pyro-GC/MS): Pyrolysis was performed on a CDS Analytical 5250-T Trapping Pyrolysis Autosampler (Oxford, Pa.) connected to a Thermo Scientific Trace 1310 gas chromatograph coupled to a Q-Exactive mass spectrometer (Waltham, Mass.). Sample vials were comprised of a quartz rod inside a quartz tube with the top headspace was packed with quartz wool. Samples were prepared with microgram quantities transferred into the vial. An initial thermal desorption step was carried out at 50° C. for 60 seconds which was sent to the GC-MS. Then a 350° C. cleaning step for 20 seconds was utilized in which all sample contents that were volatile enough were sent to an exhaust port to prevent unwanted material from reaching the column. The final step of 50° C. for 3 seconds and then ramped to 700° C. at 10 ° C./mSec and held for 60 seconds in which all material was sent to the column for analysis. Data analysis was performed using Xcalibur software version 4.1.31.9 (Thermo) and National Institute of Standards and Technology version 17 (Gaithersburg, Md.) library to help identify spectral peaks of interest.

### Studies in Mammalian Cells.

Endotoxin Assay: Pyrochrome Test Kit with glucashield reconstitution buffer and control standard endotoxin (Associates of Cape Cod Inc, East Falmouth, Mass.) were used to detect and quantify endotoxins following the manufacturer's protocol. Supernatant from PET-NP and PET-RB NP was tested in limulus amebocyte lysate (LAL) reagent water (Associates of Cape Cod Inc, East Falmouth, Mass.). The BSA solution used for washing and suspension of the particles were also tested. To ensure that the PET NPs did not interfere with the assay, positive product controls (PPC) containing a final concentration of 0.5 EU/mL were tested in parallel at the same concentration. No interference between the two PET NPs and the assay was detected.

Cell Culture: PET NP toxicity was tested on mouse alveolar macrophage cells, RAW 264.7 (ATCC® TIB-71™, ATCC, Manassas, Va.). RAW 264.7 cells were cultured in Dulbecco's Modified Eagle's Medium (Gibco, Life Technologies, Grand Island, N.Y.), supplemented with 10% fetal bovine serum (FBS) (Gibco, Life Technologies, Grand Island, N.Y.) and 100 U penicillin/streptomycin (P/S) (Gibco, Life Technologies, Grand Island, N.Y.). Cells were maintained at 37° C. in 5% humidified CO₂, at a concentration of 1×10⁴ cell/mL and passaged twice a week by washing with pre-warmed phosphate-buffered saline (PBS) (Gibco, Life Technologies, Grand Island, N.Y.). RAW 264.7 cells were used between passage numbers 41-45.

Cytotoxicity Assays: RAW264.7 were seeded out in a 96 well plate at a concentration of 1×10⁵ cells/mL and incubated for 24 hours. PET NPs suspended in fresh media were added to the cells in a two-fold dilution with concentrations between 0.0005-0.5 mg/mL. After 24 hours of NP exposure, the media was collected for lactate dehydrogenase (LDH) release measurements. LDH assay (TOX7, Sigma-Aldrich, St. Louis, Mo.) was done according to the manufacturer protocol to measure the level of LDH released to the media. Briefly, 75 μL of media was analyzed to assess cell viability as a function of cell membrane integrity. Following media collection for LDH measurements, the monolayer was washed with PBS and MTS assay was used to determine viability and metabolic activity in the cells. MTS [3-(4,5-dimethylthiazol yl)-5-(3-carboxymethoxyphenyl)-2-(4-sulfophenyl)-2H-tetrazolium] assays (CellTiter 96® AQueous One Solution Cell Proliferation Assay, Promega, Madison, Wis.) were performed according to the manufacturer protocol. Briefly, the cell reagent solution was added to the cells, and metabolic activity determined by colorimetric measurement of MTS which is reduced to colored formazan by viable, metabolic active cells. Data was expressed as percentage of their representative controls. All studies were conducted in biological duplicates and at least experimental triplicates.

Fluorescence Microscopy: Cells were seeded at a concentration of 1×10⁵ cells/mL in glass bottom Petri dishes (MatTek, Ashland, Mass.) and after 24 h were exposed to PET-RB NP for 16 h at concentrations of 0.005, 0.05, and 0.5 mg/mL. Simultaneously with PET-RB NP exposure CellLight Lysosomes-GFP*BacMam 2.0* (Life Technologies, Grand Island, N.Y.) were added to the cells to stain lysosomes at a count of 25 particles pr cell. Cells were subsequentially fixed with 3% paraformaldehyde and 0.1% glutaraldehyde for 30 minutes at room temperature. After three washes with PBS, the cells were stained with 1:200 DAPI (Life Technologies, Grand Island, N.Y.) for 15 min at room temperature. Cells were washed three times in PBS before bright field and fluorescence imaging with a 40× objective. Imaging was done using an Olympus IX71 inverted microscope with a CCD Microscopy Camera (INFINITY3-3URF, 3.0 Megapixel, CoolLED). Image processing was done using ImageJ (NIH).

Data analysis: Data are expressed as mean ±standard deviation using the software Prism (GraphPad 7.4, GraphPad Software, San Diego, Calif.). Student's t-test were used for statistical analysis and statistical significance was at P<0.05.

### Results and Discussion

### Fabrication and Characterization of PET NPs

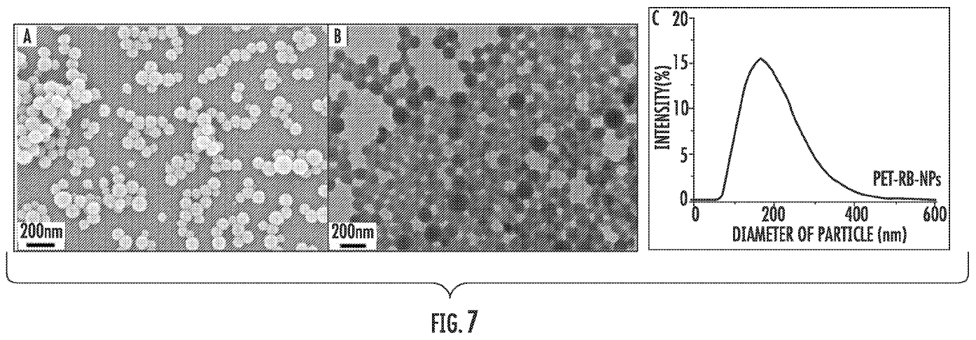

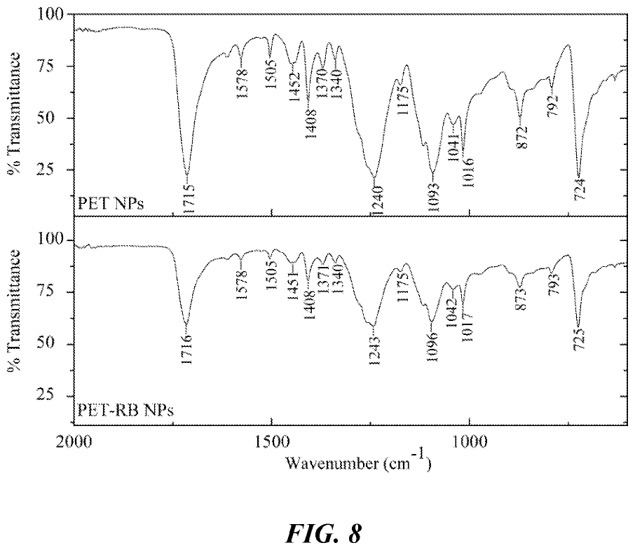

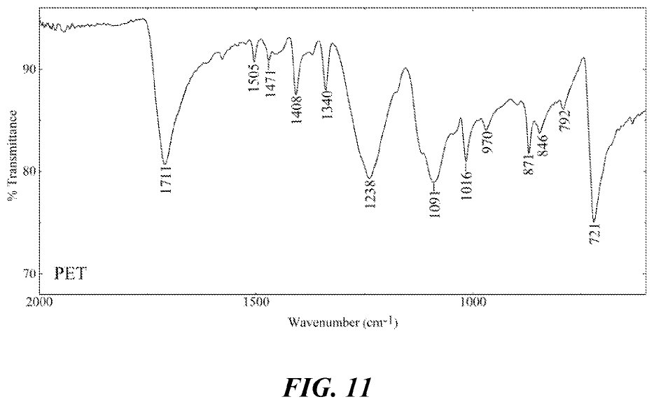

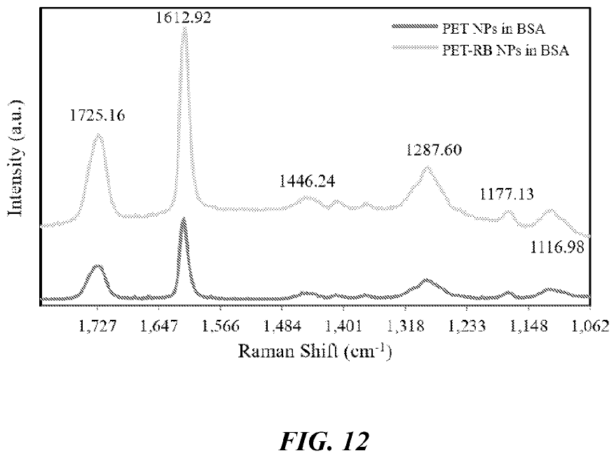

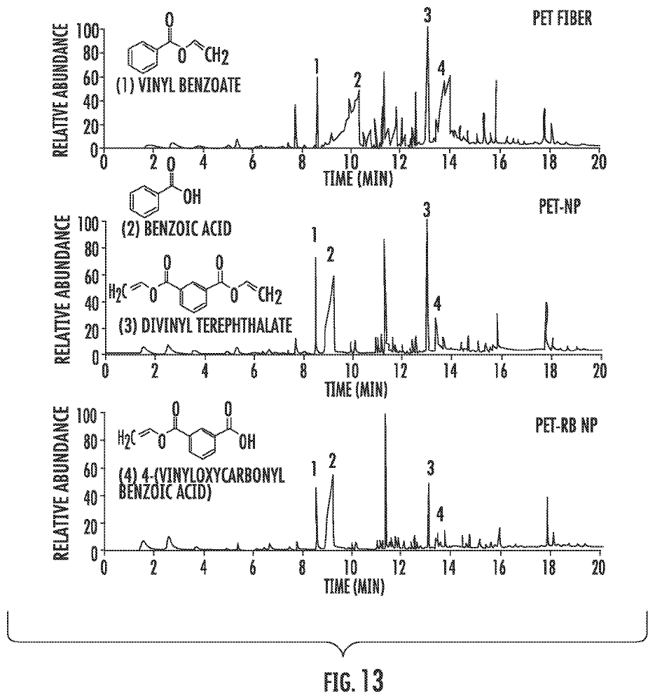

The PET NPs were fabricated with a precipitation method, wherein a solution of PET and HFIP was slowly added to ultrapure water resulting in the formation of NPs. Multiple washing steps were used to remove residual HFIP solvent from the NP formulations, resulting in an undetectable fluorine signal via ¹⁹F-NMR. While washing the PET NPs with ultrapure water, the particles aggregated and therefore a solution of BSA protein at 0.5 mg/mL was used instead to maintain particle dispersion. Here, utilization of BSA was compatible with subsequent studies in cell culture, as discussed in the following section. However, use of species-specific proteins or alternative surfactants as stabilizing agents of these NPs may be required to align with the biological system under investigation. To enable detection of the PET NPs within cells, the particles were labeled with rhodamine-B (PET-RB) by incorporation of the tracer into the NPs during fabrication. The round morphology of the PET-RB NPs was evident SEM (FIG. 7, panel A) and TEM (FIG. 7, panel B) and no morphological differences were apparent for the PET NPs without tracer (FIG. 11). After washing and resuspending the particles in a BSA solution, the hydrodynamic diameters was 170 nm±3 nm for PET-NPs and 158 nm±2 nm for PET-RB NPs, respectively (FIG. 7, panel C, FIG. 11). The washing steps with the BSA solution slightly increased the hydrodynamic diameters, as compared to the unwashed samples, but the average size distributions remained below 200 nm with polydispersity indexes at 0.2 and 0.1 for PET and PET-RB, respectively. The average diameters of NPs were also calculated from the SEM images at 95 nm±14 nm for PET NPs and 88 nm±14 nm for PET-RB NPs. The differences between the hydrodynamic diameters and the diameters calculated from SEM images is expected and could result from the existence of a BSA corona in the particle suspensions.⁴³ The zeta potential for NPs suspended within the BSA solution was −37 mV for PET NPs and −38 mV for PET-RB NPs, which supports the high dispersity and stability of the particles. For example, the PET NPs measured 164±4 nm (PDI 0.2) after one month of storage at room temperature. To explore the composition of the NPs, FT-IR analysis was performed (FIG. 8). The FT-IR profiles of NPs showed characteristic absorption bands of the PET bulk polymer (FIG. 11) and as previously reported,⁴⁴⁻⁴⁶ including at 1715 cm⁻¹ (C═O stretching), 1578 cm⁻¹ (stretching of C═C in ring), and 1505 cm⁻¹ (in-plane bending of C═H in ring; stretching of C═C in ring), 1240 cm⁻¹ (C═O in-plane bending, C═C stretching, C(═O)—O stretching)⁴⁶ and 724 cm⁻¹ (interaction of ester group and benzene ring⁴⁴). As shown in FIG. 8, the prominent IR absorption bands were similar between PET and PET-RB NPs. Interestingly, the typical bands associated with rhodamine-B, such as 1690 cm⁻¹ (C═C stretching) were absent for the PET-RB NPs, despite the verification of the fluorescent tracer with fluorescence microscopy. The absence of the rhodamine-B absorbance bands in the FT-IR could be due to the low concentration of the tracer, which was undetectable in the IR spectrum. Additional testing using Raman spectroscopy also confirmed various moieties within the PET and PET-RB NPs (FIG. 12). The main peak at 1612.92 cm⁻¹ corresponded to Raman scattering due to benzene rings in the PET structure. Other secondary peaks were located at 1725.16 cm⁻¹ (carbonyl stretching), 1446.24 and 1287.60 cm⁻¹ (weaker C—C bonds), and 1177 and 1116.98 cm⁻ (weaker C—O—C asymmetric stretching vibrations). Further analysis of the PET and PET-RB NPs was performed by pyro-GC-MS (FIG. 13).

The surface chemical states in PET NPs in BSA with and without rhodamine-B were investigated by XPS analysis. Table 1 shows the binding energies of all the elements present in the samples. The shift in the binding energies for the C 1 s, N 1 s, O 1 s, Zn 2 p and S 2 p spectra correspond to the difference in the interactions between the elements and PET structure. The peak centered at 284.4 eV for C is is present in both samples and is associated with phenyl carbons in the PET structure. A satellite peak centered around 291 eV is due to the π-π* shake-up process in the aromatic ring within the structure. The O 1s spectrum centered around 530.5 eV corresponds to C═O bond. There is also presence of a peak for N 1 s centered around 399.5 eV is the result of C—N bonding between nitrogen and the aromatic PET ring. In addition, Zn 2 p peak with two spin-orbit splits of 2 p3/2 and 2p1/2 with ˜23 eV difference in the binding energy is observed. The 2p3/2 centered at 1021.3 eV confirms the presence of Zinc in the Zn⁺² chemical environment. No noticeable shift in the binding energies is observed in both samples. Lastly, there is peaks for S 2p3/2 in S 2p spectra around 163 eV in both samples.

TABLE 1
Binding energies of major elements present in the PET samples.

| | C 1s | | | | Zn 2p | | |
|---|---|---|---|---|---|---|---|
| Sample | C-C | Satellite | O 1s | N 1s | Zn 2p_{3/2} | Zn 2p_{1/2} | S 2p |
| PET NPs in | 284.4 | 291.2 | 530.5 | 399.5 | 1021.3 | 1044.4 | 162.8 |
| BSA | | | | | | | |
| PET-RB NPs | 284.4 | 291 | 530.6 | 399.8 | 1021.1 | 1045 | 163.1 |
| in BSA | | | | | | | |

### Evaluation of PET NPs in Mammalian Cells

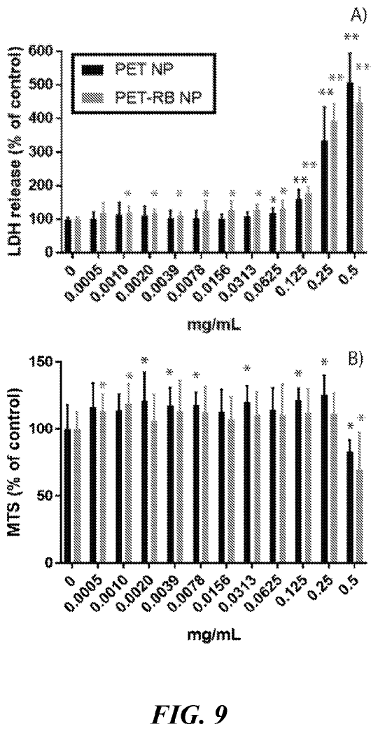

Prior to evaluation in mammalian cells, a kinetic turbidity LAL assay was used to ascertain potential endotoxin contamination of the PET NPs. Although levels of endotoxins were detectable, the values were low showing 0.1 EU/mL and 0.064 EU/mL for PET-NPs and PET-RB NPs, respectively. The cytotoxicity and uptake of PET NPs were evaluated in murine alveola macrophages, RAW264.7 in a dose-response manner. Cytotoxicity was evaluated by determining cell membrane integrity (LHD release) and metabolic activity (MTS) (FIG. 9). A significant increase in LDH release was observed at 0.0625 mg/mL for PET-NP (P-value=0.0016) and at 0.0010 mg/mL for PET-RB NP (P-value=0.0034). At the concentration of 0.125 mg/mL for both PET NPs (160±27.5% of control for PET NP and 178±18.3% of control for PET-RB NPs) the LDH release continued to increase with increasing concentration, so that LDH release for 0.5 mg/mL was 506±85% of control for PET-NP and 447±46.1% of control for PET-RB NPs. On the other hand, a slight increase in the MTS assay were observed the lowest concentration of PET NPs. Only at the highest tested concentration of PET NPs of 0.5 mg/mL did the MTS assay show a decrease in mitochondrial activity (82.9±8.77% of control for PET-NP and 71.3±29.4% of control for PET-RB NPs). Together these findings suggest that the cell membrane integrity was impacted at a lower NP concentration before mitochondrial activity was altered.

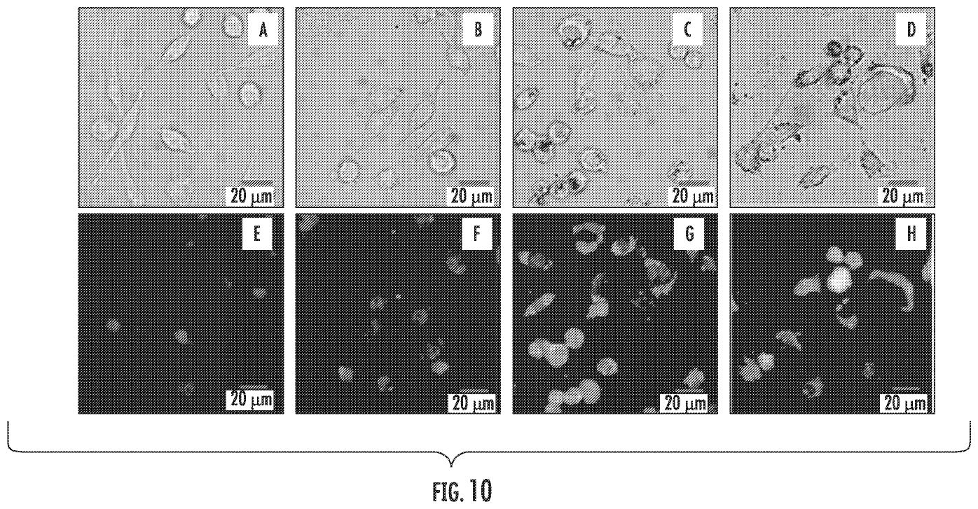

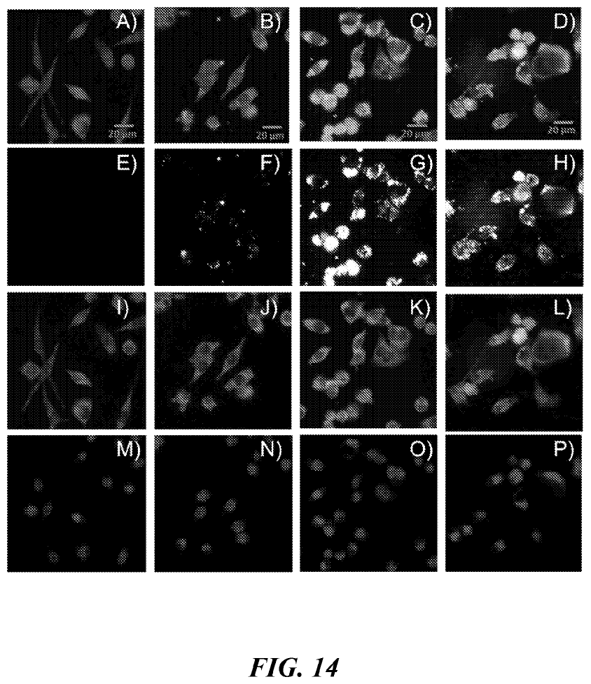

The cellular uptake of PET-RB NPs and resulting morphological changes in RAW264.7 cells were evident from bright field and fluorescence microscopy. Following exposure to a low concentration of 0.005 mg/mL PET-RB NPs, individual particles were visible in the cell cytoplasm (FIG. 10, panel B, panel F), however at concentrations of 0.05 and 0.5 mg/mL PET-RB NPs large clusters of NPs were observed intracellularly both in bright field (FIG. 10, panels A−D) and fluorescence microscopy (FIG. 10, panels E−H). The individual fluorescence channels for the fluorescence microscopy (FIG. 10, panels E−H) are shown in FIG. 14. Cell nuclei (blue channel) are shown in FIG. 14, panels M−P, cell cytoplasm (green channel) are shown in FIG. 14, panels I−L, and PET-RB NPs (red channel) are shown in FIG. 14, panels E−H. The fluorescence intensity of the larger NP aggregates oversaturated at the exposure time needed to visualize individual PET-RB NPs particles, making the aggregates look larger in the fluorescence microscopy images compare to the bright field images. Since the PET-RB NPs showed a low level of autofluorescence in the green wavelength it was not possible to determine if PET-RB NPs were associated with lysosomes. At 0.05 mg/mL PET-RB NPs, particles were observed inside phagocytic bodies, but while several cells had formed a tight phagosome around the NPs, vacuoles with large empty spaces surrounding the NPs were observed at the higher concentration. At the highest concentration, phagosomes enlarged and caused an elongated, crescent-shaped nuclei in the periphery of the cells. Morphological changes such as blebs were observed at 0.005 mg/mL, indicating cell membranes delaminate from cortical cytoskeletal structures.⁴⁷ These blebs became more numerous at 0.05 mg/mL, but not at 0.5 mg/mL. At 0.5 mg/mL a condensation and increase fluorescence intensity of the nuclei were observed supporting the cytotoxicity data, indicating that a number of cells are dead at this concentration.

### Conclusions

The environmental existence of fragmented plastics, derived from high-commodity polymers, is an emerging concern with unknown consequences in biological systems and for human health. As a crucial high-commodity polymer and contributor of plastic waste, PET has infiltrated drinking water, food, and beverages in the form of small-scale debris (i.e., microplastics), as shown in various reports. Although current reports have focused on micron-scale plastics, a potential exists for environmental contamination of nanoscale PET, as well.

PET NPs with hydrodynamic diameters below 200 nm were synthesized. To support studies in cell models, a rhodamine B fluorescent tracer was incorporated into the PET NPs and uptake within RAW264.7 macrophages was measured. The results showed uptake of PET-RB NPs in the macrophages in a dose-response manner. The findings indicated that a lower concentration of PET NPs (0.0010 mg/mL) was required to impact the integrity of the cell membrane of macrophages, as compared to concentrations of PET NPs required to alter mitochondrial activity (0.5 mg/mL). Clear morphological changes occurred at higher concentrations of PET NPs (0.5 mg/mL), showing enlarged phagosomes that caused elongation of nuclei and likely cell death. This study shows that mammalian macrophage cells are affected by PET nanoplastics.

### References, Example 4

1. Gilbert, M., Chapter 1—Plastics Materials: Introduction and Historical Development. In Brydson's Plastics Materials (Eighth Edition), Gilbert, M., Ed. Butterworth-Heinemann: 2017; pp 1-18.
2. Rochman, C. M., Microplastics research—from sink to source. Science 2018, 360 (6384),
3. Rochman, C. M., The Complex Mixture, Fate and Toxicity of Chemicals Associated with Plastic Debris in the Marine Environment. In Marine Anthropogenic Litter, Bergmann, M.; Gutow, L.; Klages, M., Eds. Springer International Publishing: Cham, 2015; pp 117-140.
4. Cózar, A.; Echevarria, F.; González-Gordillo, J. I.; Irigoien, X.; Úbeda, B.; Hernández-León, S.; Palma, Á. T.; Navarro, S.; García-de-Lomas, J.; Ruiz, A.; Fernández-de-Puelles, M. L.; Duarte, C. M., Plastic debris in the open ocean. Proceedings of the National Academy of Sciences 2014, 111 (28), 10239.
5. Ivar do Sul, J. A.; Costa, M. F., The present and future of microplastic pollution in the marine environment. Environmental Pollution 2014, 185, 352-364.
6. Eriksen, M.; Lebreton, L. C. M.; Carson, H. S.; Thiel, M.; Moore, C. J.; Borerro, J. C.; Galgani, F.; Ryan, P. G.; Reisser, J., Plastic Pollution in the World's Oceans: More than 5 Trillion Plastic Pieces Weighing over 250,000 Tons Afloat at Sea. PLoS One 2014, 9 (12), el11913-e111913.
7. Horton, A. A.; Svendsen, C.; Williams, R. J.; Spurgeon, D. J.; Lahive, E., Large microplastic particles in sediments of tributaries of the River Thames, UK - Abundance, sources and methods for effective quantification. Mar Pollut Bull 2017, 114 (1), 218-226.
8. Geyer, R.; Jambeck, J. R.; Law, K. L., Production, use, and fate of all plastics ever made. Science Advances 2017, 3 (7), e1700782.
9. Lim, H. C. A., Chapter 20—Thermoplastic Polyesters. In Brydson's Plastics Materials (Eighth Edition), Gilbert, M., Ed. Butterworth-Heinemann: 2017; pp 527-543.
10. www.plasticsinsight.com/resin-intelligence/resin-prices/polyethylene-terephthalate/.
11. Dutt, K.; Soni, R. K., A review on synthesis of value added products from polyethylene terephthalate (PET) waste. Polymer Science Series B 2013, 55 (7), 430-452.
12. Karayannidis, G. P.; Achilias, D. S., Chemical Recycling of Poly(ethylene terephthalate). Macromolecular Materials and Engineering 2007, 292 (2), 128-146.
13. Singh, B.; Sharma, N., Mechanistic implications of plastic degradation. Polymer Degradation and Stability 2008, 93 (3), 561-584.
14. Day, M.; Wiles, D. M., Photochemical decomposition mechanism of poly(ethylene terephthalate). Journal of Polymer Science Part B: Polymer Letters 1971, 9 (9), 665-669.
15. Day, M.; Wiles, D. M., Photochemical degradation of poly(ethylene terephthalate). II. Effect of wavelength and environment on the decomposition process. Journal of Applied Polymer Science 1972, 16 (1), 191-202.
16. Day, M.; Wiles, D. M., Photochemical degradation of poly(ethylene terephthalate). III. Determination of decomposition products and reaction mechanism. Journal of Applied Polymer Science 1972, 16 (1), 203-215.
17. Launay, A.; Thominette, F.; Verdu, J., Hydrolysis of poly(ethylene terephthalate): a kinetic study. Polymer Degradation and Stability 1994, 46 (3), 319-324.
18. S. Venkatachalam, S. G. N., Jayprakash V. Labde, Prashant R. Gharal, Krishna Rao and Anil K. Kelkar Degradation and Recyclability of Poly (Ethylene Terephthalate), Polyester. In Polyester, Saleh, H. E.-D. M., Ed. IntechOpen: 2012.
19. Oβmann, B. E.; Sarau, G.; Holtmannspotter, H.; Pischetsrieder, M.; Christiansen, S. H.; Dicke, W., Small-sized microplastics and pigmented particles in bottled mineral water. Water Research 2018, 141, 307-316.
20. Pivokonsky, M.; Cermakova, L.; Novotna, K.; Peer, P.; Cajthaml, T.; Janda, V., Occurrence of microplastics in raw and treated drinking water. Science of The Total Environment 2018, 643, 1644-1651.
21. Schymanski, D.; Goldbeck, C.; Humpf, H.-U.; Furst, P., Analysis of microplastics in water by micro-Raman spectroscopy: Release of plastic particles from different packaging into mineral water. Water Research 2018, 129, 154-162.
22. Liebezeit, G.; Liebezeit, E., Synthetic particles as contaminants in German beers. Food Additives & Contaminants: Part A 2014, 31 (9), 1574-1578.
23. Koelmans, A. A.; Mohamed Nor, N. H.; Hermsen, E.; Kooi, M.; Mintenig, S. M.; De France, J., Microplastics in freshwaters and drinking water: Critical review and assessment of data quality. Water Research 2019, 155, 410-422.
24. Iñiguez, M. E.; Conesa, J. A.; Fullana, A., Microplastics in Spanish Table Salt. Scientific Reports 2017, 7 (1), 8620.
25. Fischer, M.; Goβmann, I.; Scholz-Böttcher, B. M., Fleur de Sel—An interregional monitor for microplastics mass load and composition in European coastal waters? Journal of Analytical and Applied Pyrolysis 2019, 144, 104711.
26. Van Cauwenberghe, L.; Janssen, C. R., Microplastics in bivalves cultured for human consumption. Environmental Pollution 2014, 193, 65-70.
27. Barboza, L. G. A.; Lopes, C.; Oliveira, P.; Bessa, F.; Otero, V.; Henriques, B.; Raimundo, J.; Caetano, M.; Vale, C.; Guilhermino, L., Microplastics in wild fish from North East Atlantic Ocean and its potential for causing neurotoxic effects, lipid oxidative damage, and human health risks associated with ingestion exposure. Science of The Total Environment 2020, 717, 134625.
28. Eriksen, M.; Maximenko, N.; Thiel, M.; Cummins, A.; Lattin, G.; Wilson, S.; Hafner, J.; Zellers, A.; Rifman, S., Plastic pollution in the South Pacific subtropical gyre. Mar Pollut Bull 2013, 68 (1), 71-76.
29. Guo, X.; Wang, J., The chemical behaviors of microplastics in marine environment: A review. Mar Pollut Bull 2019, 142, 1-14.
30. Browne, M. A.; Crump, P.; Niven, S. J.; Teuten, E.; Tonkin, A.; Galloway, T.; Thompson, R., Accumulation of Microplastic on Shorelines Woldwide: Sources and Sinks. Environmental Science & Technology 2011, 45 (21), 9175-9179.
31. Kole, P. J.; Löhr, A. J.; Van Belleghem, F. G. A. J.; Ragas, A. M. J., Wear and Tear of Tyres: A Stealthy Source of Microplastics in the Environment. International Journal of Environmental Research and Public Health 2017, 14 (10), 1265.
32. Andrady, A. L., Microplastics in the marine environment. Mar Pollut Bull 2011, 62 (8), 1596-1605.
33. Hartmann, N. B.; Hüffer, T.; Thompson, R. C.; Hassellöv, M.; Verschoor, A.; Daugaard, A. E.; Rist, S.; Karlsson, T.; Brennholt, N.; Cole, M.; Herrling, M. P.; Hess, M. C.; Ivleva, N. P.; Lusher, A. L.; Wagner, M., Are We Speaking the Same Language? Recommendations for a Definition and Categorization Framework for Plastic Debris. Environmental Science & Technology 2019, 53 (3), 1039-1047.
34. Verschoor, A. J. Towards a definition of microplastics: Considerations for the specification of physico-chemical properties; 2015.
35. Arthur, C., Baker, J., Bamford, H. In Proceedings of the International Research Workshop on Microplastic Marine Debris, NOAA Technical Memorandum NOS-OR&R-30:2009.
36. Bouwmeester, H.; Hollman, P. C. H.; Peters, R. J. B., Potential Health Impact of Environmentally Released Micro- and Nanoplastics in the Human Food Production Chain: Experiences from Nanotoxicology. Environmental Science & Technology 2015, 49 (15), 8932-8947.
37. Hüffer, T.; Weniger, A.-K.; Hofmann, T., Sorption of organic compounds by aged polystyrene microplastic particles. Environmental Pollution 2018, 236, 218-225.
38. Xia, T.; Kovochich, M.; Liong, M.; Zink, J. I.; Nel, A. E., Cationic Polystyrene Nanosphere Toxicity Depends on Cell-Specific Endocytic and Mitochondrial Injury Pathways. ACS Nano 2008, 2 (1), 85-96.
39. Behzadi, S.; Serpooshan, V.; Tao, W.; Hamaly, M. A.; Alkawareek, M. Y.; Dreaden, E. C.; Brown, D.; Alkilany, A. M.; Farokhzad, 0. C.; Mahmoudi, M., Cellular uptake of nanoparticles: journey inside the cell. Chemical Society Reviews 2017, 46 (14), 4218-4244.
40. Magri, D.; Sánchez-Moreno, P.; Caputo, G.; Gatto, F.; Veronesi, M.; Bardi, G.; Catelani, T.; Guarnieri, D.; Athanassiou, A.; Pompa, P. P.; Fragouli, D., Laser Ablation as a Versatile Tool To Mimic Polyethylene Terephthalate Nanoplastic Pollutants: Characterization and Toxicology Assessment. ACS Nano 2018, 12 (8), 7690-7700.
41. Bauers, F. M.; Thomann, R.; Mecking, S., Submicron Polyethylene Particles from Catalytic Emulsion Polymerization. Journal of the American Chemical Society 2003, 125 (29), 8838-8840.
42. Rodríguez-Hernández, A. G.; Muñoz-Tabares, J. A.; Aguilar-Guzmán, J. C.; Vazquez-Duhalt, R., A novel and simple method for polyethylene terephthalate (PET) nanoparticle production. Environmental Science: Nano 2019, 6 (7), 2031-2036.
43. Kokkinopoulou, M.; Simon, J.; Landfester, K.; Mailander, V.; Lieberwirth, I., Visualization of the protein corona: towards a biomolecular understanding of nanoparticle-cell-interactions. Nanoscale 2017, 9 (25), 8858-8870.
44. Edge, M.; Wiles, R.; Allen, N. S.; McDonald, W. A.; Mortlock, S. V., Characterisation of the species responsible for yellowing in melt degraded aromatic polyesters—I: Yellowing of poly(ethylene terephthalate). Polymer Degradation and Stability 1996, 53 (2), 141-151.
45. Pereira, A. P. d. S.; Silva, M. H. P. d.; Lima Júnior, É. P.; Paula, A. d. S.; Tommasini, F. J., Processing and Characterization of PET Composites Reinforced with Geopolymer Concrete Waste. Materials Research 2017, 20, 411-420.
46. Donelli, I.; Freddi, G.; Nierstrasz, V. A.; Taddei, P., Surface structure and properties of poly-(ethylene terephthalate) hydrolyzed by alkali and cutinase. Polymer Degradation and Stability 2010, 95 (9), 1542-1550.
47. Olson, M.; Julian, L., Apoptotic membrane dynamics in health and disease. Cell Health and Cytoskeleton 2015, 7, 133.

Although various features of the invention may be described in the context of a single embodiment, the features may also be provided separately or in any suitable combination. Conversely, although the invention may be described herein in the context of separate embodiments for clarity, the invention may also be implemented in a single embodiment.

The foregoing is illustrative of the present inventive concept and is not to be construed as limiting thereof. Further embodiments of the inventive concept are exemplified in the following claims, which equivalents of the claims are to be included therein.

1. A nanoplastic or microplastic particle comprising:
a nanoplastic or microplastic polymer, polymer composite, or polymer matrix; and
a fluorescent tag or a radioactive tag.

2. The nanoplastic or microplastic particle of claim 1, wherein the microplastic polymer, polymer composite, or polymer matrix is at least one of polyethylene terephthalate (PET), polyethylene (PE), high density PE (HDPE), low density PE (LDPE), linear-low-density polyethylene (LLDPE), polyvinyl chloride (PVC), polypropylene (PP), polystyrene (PS), polylactic acid (PLA), polycarbonate (PC) polymethyl methacrylate (PMMA), Polyamide (PA), polyacrylic acid (PAA), polyacrylonitrile (PAN), polyoxymethylene (POM), polyurethane (PUR), silicone, nylon, or acrylonitrile butadiene styrene (ABS).

3. The nanoplastic or microplastic particle of claim 1, wherein the nanoplastic or microplastic particle comprises a fluorescent tag, wherein the fluorescent tag is at least one of rhodamine, fluorescein, Alexa-Fluor compounds, Nile Red, R-Phycoerthyrin, Pacific Blue, Cascade Blue, Texas Red, Cy5, Cy3, Cy7, hydroxycoumarin, aminocourmarin, or methoxycoumarin.

4. The nanoplastic or microplastic particle of claim 1, wherein the nanoplastic or microplastic particle comprises a fluorescent tag, wherein the fluorescent tag comprises a bioconjugate.

5. The nanoplastic or microplastic particle of claim 1, wherein the nanoplastic or microplastic particle comprises a polymer matrix and a fluorescent tag, wherein the fluorescent tag is distributed throughout the polymer matrix.

6. The nanoplastic or microplastic particle of claim 1, wherein the nanoplastic or microplastic particle comprises a functionalized surface and a fluorescent tag, wherein the fluorescent tag is associated with the functionalized surface.

7. The nanoplastic or microplastic particle of claim 6, wherein the functionalized surface comprises chemical groups selected from the group consisting of —COOH, —COO⁻, —NH₃⁺, —NH₂, —OH, PEG, streptavidin, a streptavidin-biotin complex, and an antibody conjugate.

8. The nanoplastic or microplastic particle of claim 1, wherein particle size is less than about one micron.

9. The nanoplastic or microplastic particle of claim 1, wherein particle size is less than about 500 nm.

10. The nanoplastic or microplastic particle of claim 1, wherein particle size is less than about 100 nm.

11. A reference standard material comprising nanoplastic or microplastic particles, the nanoplastic or microplastic particles comprising:
a nanoplastic or microplastic polymer, polymer composite, or polymer matrix; and
a fluorescent tag or a radioactive tag.

12. The reference standard material of claim 11, wherein the nanoplastic or microplastic particles are sized to represent particle size distributions found environmentally.

13-34. (canceled)

35. A method of preparing nanoplastic and/or microplastic particles comprising:
dissolving a plastic in a first solvent to provide a plastic solution;
precipitating the plastic solution in a second solvent; and
evaporating the first solvent to provide a dispersion of nanoplastic or microplastic particles in the second solvent.

36. The method of claim 35, wherein the plastic is selected from the group consisting of polyethylene terephthalate (PET), polyethylene (PE), high density PE (HDPE), low density PE (LDPE), linear-low-density polyethylene (LLDPE), polyvinyl chloride (PVC), polypropylene (PP), polystyrene (PS), polylactic acid (PLA), polycarbonate (PC) polymethyl methacrylate (PMMA), Polyamide (PA), polyacrylic acid (PAA), polyacrylonitrile (PAN), polyoxymethylene (POM), polyurethane (PUR), silicone, nylon, and acrylonitrile butadiene styrene (ABS).

37. The method of claim 36, wherein the plastic is PET.

38. The method of claim 35, wherein the first solvent is selected from the group consisting of phenol, DMSO, nitrobenzene, o-chlorophenol, o-cresol, diphenylamine, dichloromethane, and HFIP, or any combination thereof.

39. The method of claim 35, wherein the first solvent is hexafluoroisopropanol (HFIP).

40. The method of claim 35, wherein the plastic solution comprises the plastic in an amount between about 0.1 and about 5 weight %.

41. The method of claim 38, wherein the second solvent is water.

42. The method of claim 35, wherein the plastic solution is precipitated in the second solvent by adding the plastic solution to the second solvent at a rate between 0.1 and 5 mL/min.

43. The method of claim 42, wherein the plastic solution is precipitated in the second solvent by adding the plastic solution to the second solvent second solvent at a rate of about 1 mL/min.

44. The method of claim 35, wherein the plastic solution has a volume of about 10 mL and the second solvent has a volume of between about 50 mL and about 5000 mL

45. The method of claim 35, wherein the second solvent has a temperature of between about 0° C. and about 20° C.

46. The method of claim 35, wherein the plastic is dissolved in the first solvent with a fluorescent tag.

47. The method of claim 46, wherein the fluorescent tag is selected from the group consisting of rhodamine, fluorescein, Alexa-Fluor compounds, Nile Red, R-Phycoerthyrin, Pacific Blue, Cascade Blue, Texas Red, Cy5, Cy3, Cy7, hydroxycoumarin, aminocourmarin, and methoxycoumarin.

48. The method of claim 35, wherein the nanoplastic or microplastic particles prepared have an average size less than about a micron.

49. The method of claim 35, wherein the nanoplastic or microplastic particles prepared have an average size less than about 500 nm.

50. The method of claim 35, wherein the nanoplastic or microplastic particles prepared have an average size less than about 150 nm.

51. The method of claim 35, wherein the nanoplastic or microplastic particles prepared have an average size less than about 100 nm.
